(19) United States
(12) Patent Application Publication
(10) Pub. No.: US 2025/0107591 A1
(43) Pub. Date: Apr. 3, 2025

(54) A SOLE AND METHOD OF DETERMINING ATTRIBUTES THEREOF

(71) Applicant: The EduPod Pty Ltd, Brisbane (AU)

(72) Inventor: Jason MCLELLAN, Brisbane (AU)

(21) Appl. No.: 18/684,387

(22) PCT Filed: Aug. 16, 2022
(86) PCT No.: PCT/AU2022/050897

§ 371 Date: Feb. 16, 2024

(30) Foreign Application Priority Data
Aug. 17, 2021 (AU) 2021105690
Aug. 17, 2021 (AU) 2021218050

(51) Int. Cl. A43B 7/16

(52) U.S. Cl. CPC A43B 7/16

(57) ABSTRACT

A method of determining an attribute of a sole, the method including the steps of: determining a talar head palpation of a user's foot and assigning a value based on the position of the talus head, determining a supra and infra lateral malleoli curvature value of the user's foot and assigning a value based on the prominence of the infra and supra malleolar curves, determining a calcaneal frontal plane position value of the user's foot and assigning a value based on an angle of the calcaneus, determining a prominence of the talo-navicular joint value of the user's foot and assigning a value based on the curvature of the talo-navicular joint, determining a congruence of medial longitudinal arch value of the user's foot and assigning a value based on the curvature of the arch, determining an abduction/adduction of forefoot on rearfoot value of the user's foot and assigning a value based on the alignment, determining a minimum possible sum of the values and a maximum possible sum of the values and dividing the range of values into a predetermined number of bands from the minimum possible sum to the maximum possible sum, wherein the attribute is dependent on which band the sum of the assigned values fall into.

### FIELD OF INVENTION

The present invention relates to a sole. The present invention has particular but not exclusive application for determining attributes of a sole. The patent specification describes this use but it is by way of example only and the invention is not limited to this use.

### BACKGROUND OF THE INVENTION

Mass produced footwear has been around for a long time. A benefit of mass produced footwear is that it can be easy to find a pair of shoes for a user. However, a drawback with mass produced footwear is that users with certain foot conditions cannot wear mass produced footwear.

Typically users with conditions such as mild to severe planus (flat) feet, and mild to severe cavus (high arched) feet may require custom made footwear. In some cases, custom made soles can be made which can be inserted into mass produced footwear.

A problem with custom made footwear or custom made soles is that it can be prohibitively expensive for users compared to mass produced footwear.

Another problem with custom made footwear or custom made soles is that it can take a significant amount of time to produce the footwear or soles.

A further problem with custom made footwear or custom made soles is that the custom made footwear or the custom made soles are typically unique for each user.

### OBJECT OF THE INVENTION

It is an object of the present invention to overcome or at least alleviate one or more of the above mentioned problems with custom made soles and/or provide the consumer with a useful or commercial choice.

### SUMMARY OF THE INVENTION

In one aspect, the present invention broadly resides in a method of determining one or more attributes of a sole, the method including the steps of:

determining a talar head palpation of a user's foot and assigning a value based on the position of the talus head;

determining a supra and infra lateral malleoli curvature of the user's foot and assigning a value based on the prominence of the infra and supra malleolar curves;

determining a calcaneal frontal plane position of the user's foot and assigning a value based on an angle of the calcaneus;

determining a prominence of the talo-navicular joint of the user's foot and assigning a value based on the curvature of the talo-navicular joint;

determining a congruence of medial longitudinal arch of the user's foot and assigning a value based on the curvature of the arch;

determining an abduction/adduction of forefoot on rearfoot of the user's foot and assigning a value based on the alignment; and

determining a minimum possible sum of the values and a maximum possible sum of the values and dividing the range of values into a predetermined number of bands from the minimum possible sum to the maximum possible sum, wherein the one or more attributes is dependent on which band the sum of the assigned values fall into.

Preferably the predetermined number of bands is six bands.

In another aspect, the present invention broadly resides in a method of determining one or more attributes of a sole, the method including the steps of:

determining a talar head palpation of a user's foot and assigning a value based on the position of the talus head;

determining a supra and infra lateral malleoli curvature of the user's foot and assigning a value based on the prominence of the infra and supra malleolar curves;

determining a calcaneal frontal plane position of the user's foot and assigning a value based on an angle of the calcaneus;

determining a prominence of the talo-navicular joint of the user's foot and assigning a value based on the curvature of the talo-navicular joint;

determining a congruence of medial longitudinal arch of the user's foot and assigning a value based on the curvature of the arch;

determining an abduction/adduction of forefoot on rearfoot of the user's foot and assigning a value based on the alignment; and

determining a minimum possible sum of the values and a maximum possible sum of the values and dividing the range of values into six bands from the minimum possible sum to the maximum possible sum, wherein the one or more attributes is dependent on which band the sum of the assigned values fall into.

It will be appreciated that dividing the range of values into six bands from the minimum possible sum to the maximum possible sum may also be referred to as applying the range of values into six bands from the minimum possible sum to the maximum possible sum.

Preferably one of the one or more attributes is a rear foot posting. Preferably the rear foot posting varies between a 4° valgus foot position and a 4° varus foot position dependent on which band the sum of the assigned values fall into. Preferably the rear foot posting is 4° valgus foot position if the sum of the assigned values falls into the first band. Preferably the rear foot posting is 3° valgus foot position if the sum of the assigned values falls into the second band. Preferably the rear foot posting is 2° valgus foot position if the sum of the assigned values falls into the third band. Preferably the rear foot posting is 0° if the sum of the assigned values falls into the fourth band. Preferably the rear foot posting is 3° varus foot position if the sum of the assigned values falls into the fifth band. Preferably the rear foot posting is 4° varus foot position if the sum of the assigned values falls into the sixth band.

Preferably one of the one or more attributes is a j-pod position. Preferably the j-pod position varies between extending high laterally and extending high medially dependent on which band the sum of the assigned values fall into. Preferably the j-pod position extends high laterally if the sum of the assigned values falls into the first band. Preferably the j-pod position extends high laterally if the sum of the assigned values falls into the second band. Preferably the j-pod position extends high laterally if the sum of the assigned values falls into the third band. Preferably the j-pod position is neutral if the sum of the assigned values falls into the fourth band. Preferably the j-pod position extends high medially if the sum of the assigned values falls into the fifth band. Preferably the j-pod position extends high medially if the sum of the assigned values falls into the sixth band.

Preferably one of the one or more attributes is a functional heel raise. Preferably the functional heel raise varies between 0 mm and 6 mm dependent on which band the sum of the assigned values fall into. Preferably the functional heel raise is 6 mm if the sum of the assigned values falls into the first band. Preferably the functional heel raise is 4 mm if the sum of the assigned values falls into the second band. Preferably the functional heel raise is 4 mm if the sum of the assigned values falls into the third band. Preferably the functional heel raise is 0 mm if the sum of the assigned values falls into the fourth band. Preferably the functional heel raise is 4 mm if the sum of the assigned values falls into the fifth band. Preferably the functional heel raise is 6 mm if the sum of the assigned values falls into the sixth band.

Preferably the method includes providing a sole with one or more of the one or more attributes.

In one embodiment, the method includes making a sole with one or more of the one or more attributes.

Preferably the sole has an inherent heel raise of between 10 mm and 25 mm. More preferably the sole has an inherent heel raise of between 15 mm and 20 mm.

Preferably the sole has a rocker radii angle of between 120 mm and 200 mm. More preferably the sole has a rocker radii angle of substantially 160 mm.

Preferably the sole has a rocker radii position of between 40 to 70 percent of the sole length. More preferably the sole has a rocker radii position of substantially 60 percent of the sole length.

Preferably the sole has a Shore A hardness of between 25 to 65.

Preferably a top surface of the sole has a general angle of 2° to 10° relative to a flat ground surface.

In another embodiment, the method includes providing footwear with one or more of the one or more attributes.

In one embodiment, the method includes making footwear with one or more of the one or more attributes.

It will be understood that in the embodiments including footwear, the footwear preferably includes the sole.

Preferably the footwear has an inherent heel raise of between 10 mm and 25 mm. More preferably the footwear has an inherent heel raise of between 15 mm and 20 mm.

Preferably the footwear has a rocker radii angle of between 120 mm and 200 mm. More preferably the footwear has a rocker radii angle of substantially 160 mm.

Preferably the footwear has a rocker radii position of between 40 to 70 percent of the sole length. More preferably the footwear has a rocker radii position of substantially 60 percent of the sole length.

In one embodiment, the method further includes 3d printing a sole with one or more of the one or more attributes.

In one embodiment the step of determining a talar head palpation of a user's foot and assigning a value based on the position of the talus head includes using augmented reality. In another embodiment, the step of determining a talar head palpation of a user's foot and assigning a value based on the position of the talus head includes using machine learning to assign the value. Preferably the value is assigned using a scan or photo of the user's foot.

In one embodiment the step of determining a supra and infra lateral malleoli curvature of the user's foot and assigning a value based on the prominence of the infra and supra malleolar curves includes using augmented reality. In another embodiment, the step of determining a supra and infra lateral malleoli curvature of the user's foot and assigning a value based on the prominence of the infra and supra malleolar curves includes using machine learning to assign the value. Preferably the value is assigned using a scan or photo of the user's foot.

In one embodiment the step of determining a calcaneal frontal plane position of the user's foot and assigning a value based on an angle of the calcaneus includes using augmented reality. In another embodiment, the step of determining a calcaneal frontal plane position of the user's foot and assigning a value based on an angle of the calcaneus includes using machine learning to assign the value. Preferably the value is assigned using a scan or photo of the user's foot.

In one embodiment the step of determining a prominence of the talo-navicular joint of the user's foot and assigning a value based on the curvature of the talo-navicular joint includes using augmented reality. In another embodiment, the step of determining a prominence of the talo-navicular joint of the user's foot and assigning a value based on the curvature of the talo-navicular joint includes using machine learning to assign the value. Preferably the value is assigned using a scan or photo of the user's foot.

In one embodiment the step of determining a congruence of medial longitudinal arch of the user's foot and assigning a value based on the curvature of the arch includes using augmented reality. In another embodiment, the step of determining a congruence of medial longitudinal arch of the user's foot and assigning a value based on the curvature of the arch includes using machine learning to assign the value. Preferably the value is assigned using a scan or photo of the user's foot. In one embodiment the step of determining an abduction/adduction of forefoot on rearfoot of the user's foot and assigning a valued based on the alignment includes using augmented reality. In another embodiment, the step of determining an abduction/adduction of forefoot on rearfoot of the user's foot and assigning a valued based on the alignment includes using machine learning to assign the value. Preferably the value is assigned using a scan or photo of the user's foot.

Preferably, if the position of talus head is palpable on the lateral of the foot but not on the medial side, then the talar head palpation value receives a first value.

Preferably, if the talar head is felt on the lateral of the foot and only slightly palpable on the medial side of the foot, then the talar head palpation value receives a second value. Preferably the second value is higher than the first value.

Preferably, if the talar head is equally palpable on the lateral and medial side of the foot, then the talar head palpation value receives a third value. Preferably the third value is higher than the second value.

Preferably, if the talar head is slightly palpable on lateral side and palpable on medial side, then the talar head palpation value receives a fourth value. Preferably the fourth value is higher than the third value.

Preferably, if the talar head is not palpable on lateral side, but palpable on medial side, then the talar head palpation value receives a fifth value. Preferably the fifth value is higher than the fourth value.

It will be understood that the above mentioned attributes and how to determine them are clear to a person skilled in the art.

Preferably, if the curve below the malleolus is either straight or convex, then the supra and infra lateral malleoli curvature value receives a first value.

Preferably, if the curve below the malleolus is concave, but flatter or more than the curve above the malleolus, then the supra and infra lateral malleoli curvature value receives a second value. Preferably the second value is higher than the first value.

Preferably, if both the infra and supra malleolar curves are substantially equal, then the supra and infra lateral malleoli curvature value receives a third value. Preferably the third value is higher than the second value.

Preferably, if the curve below the malleolus is more concave than the curve above the malleolus, then the supra and infra lateral malleoli curvature value receives a fourth value. Preferably the fourth value is higher than the third value.

Preferably, if the curve below the malleolus is markedly more concave than the curve above the malleolus, then the supra and infra lateral malleoli curvature value receives a fifth value. Preferably the fifth value is higher than the fourth value.

It will be understood that the above mentioned attributes and how to determine them are clear to a person skilled in the art.

Preferably, if the calcaneus is more than 5° inverted, then the calcaneal frontal plane position value receives a first value.

Preferably, if the calcaneus is between vertical and 5° inverted, then the calcaneal frontal plane position value receives a second value. Preferably the second value is higher than the first value.

Preferably, if the calcaneus is substantially vertical, then the calcaneal frontal plane position value receives a third value. Preferably the third value is higher than the second value.

Preferably, if the calcaneus is between vertical and 5° everted, then the calcaneal frontal plane position value receives a fourth value. Preferably the fourth value is higher than the third value.

Preferably, if the calcaneus is more than 5° everted, then the calcaneal frontal plane position value receives a fifth value. Preferably the fifth value is higher than the fourth value.

It will be understood that the above mentioned attributes and how to determine them are clear to a person skilled in the art.

Preferably, if the area of the talo-navicular joint is markedly concave, then the prominence of the talo-navicular joint value receives a first value.

Preferably, if the area of the talo-navicular joint is slightly but definitely concave, then the prominence of the talo-navicular joint value receives a second value. Preferably the second value is higher than the first value.

Preferably, if the area of the talo-navicular joint is significantly flat, then the prominence of the talo-navicular joint value receives a third value. Preferably the third value is higher than the second value.

Preferably, if the area of the talo-navicular joint is bulging slightly, then the prominence of the talo-navicular joint value receives a fourth value. Preferably the fourth value is higher than the third value.

Preferably, if the area of the talo-navicular joint is bulging markedly, then the prominence of the talo-navicular joint value receives a fifth value. Preferably the fifth value is higher than the fourth value.

It will be understood that the above mentioned attributes and how to determine them are clear to a person skilled in the art.

Preferably, if the arch is high and acutely angled towards the posterior end of the medial arch, then the congruence of medial longitudinal arch value receives a first value.

Preferably, if the arch is moderately high and slightly acute posteriorly, then the congruence of medial longitudinal arch value receives a second value. Preferably the second value is higher than the first value.

Preferably, if the arch height is normal and concentrically curved, then the congruence of medial longitudinal arch value receives a third value. Preferably the third value is higher than the second value.

Preferably, if the arch is lowered with some flattening in the central position, then the congruence of medial longitudinal arch value receives a fourth value. Preferably the fourth value is higher than the third value.

Preferably, if the arch very low with severe flattening in the central portion and the arch makes ground contact, then the congruence of medial longitudinal arch value receives a fifth value. Preferably the fifth value is higher than the fourth value.

It will be understood that the above mentioned attributes and how to determine them are clear to a person skilled in the art.

Preferably, if there are no lateral toes visible and medial toes are clearly visible, then the abduction/adduction of forefoot on rearfoot value receives a first value.

Preferably, if the medial toes are clearly more visible than the lateral toes, then the abduction/adduction of forefoot on rearfoot value receives a second value. Preferably the second value is higher than the first value.

Preferably, if the medial and lateral toes are equally visible, then the abduction/adduction of forefoot on rearfoot value receives a third value. Preferably the third value is higher than the second value.

Preferably, if the lateral toes are clearly more visible than the medial toes, then the abduction/adduction of forefoot on rearfoot value receives a fourth value. Preferably the fourth value is higher than the third value.

Preferably, if no medial toes are visible and the lateral toes are clearly visible, then the abduction/adduction of forefoot on rearfoot value receives a fifth value. Preferably the fifth value is higher than the fourth value.

It will be understood that the above mentioned attributes and how to determine them are clear to a person skilled in the art.

Preferably when determining a value for a feature of a user's foot, the value is one of five values assigned from one extreme condition for a feature to another extreme condition for a feature. Preferably, the values range from a first extreme condition, to a first moderate condition, to a neutral condition to a second moderate condition to a second extreme condition. In one embodiment, the values substantially correspond to different bands of a bell curve distribution for conditions. Preferably machine learning utilises a bell curve distribution or the like to assign values.

Preferably, if the method utilises augmented reality, a visual of a range of conditions from a plurality of ranges can be overlaid over the user's foot and a corresponding value can be assigned depending on which range the condition of the user's foot corresponds to.

In another aspect, the present invention broadly resides in a method of determining a rear foot posting of a sole, the method including the steps of:

determining a talar head palpation of a user's foot, wherein:

if the position of talus head is palpable on the lateral of the foot but not on the medial side, then the talar head palpation value receives a relative value of −2;

if the talar head is felt on the lateral of the foot and only slightly palpable on the medial side of the foot, then the talar head palpation value receives a relative value of −1;

if the talar head is equally palpable on the lateral and medial side of the foot, then the talar head palpation value receives a relative value of 0;

if the talar head is slightly palpable on lateral side and palpable on medial side, then the talar head palpation value receives a relative value of 1;

if the talar head is not palpable on lateral side, but palpable on medial side, then the talar head palpation value receives a relative value of 2;

determining a supra and infra lateral malleoli curvature value of the user's foot, wherein:

if the curve below the malleolus is either straight or convex, then the supra and infra lateral malleoli curvature value receives a relative value of −2;

if the curve below the malleolus is concave, but flatter or more than the curve above the malleolus, then the supra and infra lateral malleoli curvature value receives a relative value of −1;

if both the infra and supra malleolar curves are substantially equal, then the supra and infra lateral malleoli curvature value receives a relative value of 0;

if the curve below the malleolus is more concave than the curve above the malleolus, then the supra and infra lateral malleoli curvature value receives a relative value of 1;

if the curve below the malleolus is markedly more concave than the curve above the malleolus, then the supra and infra lateral malleoli curvature value receives a relative value of 2;

determining a calcaneal frontal plane position value of the user's foot, wherein: if the calcaneus is more than 5° inverted, then the calcaneal frontal plane

position value receives a relative value of −2;

if the calcaneus is between vertical and 5° inverted, then the calcaneal frontal plane position value receives a relative value of −1;

if the calcaneus is substantially vertical, then the calcaneal frontal plane position value receives a relative value of 0;

if the calcaneus is between vertical and 5° everted, then the calcaneal frontal plane position value receives a relative value of 1;

if the calcaneus is more than 5° everted, then the calcaneal frontal plane position value receives a relative value of 2;

determining a prominence of the talo-navicular joint value of the user's foot, wherein:

if the area of the talo-navicular joint is markedly concave, then the prominence of the talo-navicular joint value receives a relative value of −2;

if the area of the talo-navicular joint is slightly but definitely concave, then the prominence of the talo-navicular joint value receives a relative value of −1;

if the area of the talo-navicular joint is significantly flat, then the prominence of the talo-navicular joint value receives a relative value of 0;

if the area of the talo-navicular joint is bulging slightly, then the prominence of the talo-navicular joint value receives a relative value of 1;

if the area of the talo-navicular joint is bulging markedly, then the

prominence of the talo-navicular joint value receives a relative value of 2; determining a congruence of medial longitudinal arch value of the user's foot, wherein:

if the arch is high and acutely angled towards the posterior end of the medial arch, then the congruence of medial longitudinal arch value receives a relative value of −2;

if the arch is moderately high and slightly acute posteriorly, then the congruence of medial longitudinal arch value receives a relative value of −1;

if the arch height is normal and concentrically curved, then the congruence of medial longitudinal arch value receives a relative value of 0;

if the arch is lowered with some flattening in the central position, then the congruence of medial longitudinal arch value receives a relative value of 1;

if the arch very low with severe flattening in the central portion and the arch makes ground contact, then the congruence of medial longitudinal arch value receives a relative value of 2;

determining an abduction/adduction of forefoot on rearfoot value of the user's foot, by viewing the foot from behind, wherein:

if there are no lateral toes visible and medial toes are clearly visible, then the abduction/adduction of forefoot on rearfoot value receives a relative value of −2;

if the medial toes are clearly more visible than the lateral toes, then the abduction/adduction of forefoot on rearfoot value receives a relative value of −1;

if the medial and lateral toes are equally visible, then the abduction/adduction of forefoot on rearfoot value receives a relative value of 0;

if the lateral toes are clearly more visible than the medial toes, then the abduction/adduction of forefoot on rearfoot value receives a relative value of 1; if no medial toes are visible and the lateral toes are clearly visible, then

the abduction/adduction of forefoot on rearfoot value receives a relative value

of 2;

wherein

if the talar head palpation value, the infra lateral malleoli curvature value, the 5 calcaneal frontal plane position value, the prominence of the talo-navicular joint value, the congruence of medial longitudinal arch value, and the abduction/adduction of forefoot on rearfoot value add up to between 0 and 4, then the rear foot posting is 0°; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the rear foot posting is 2° valgus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the rear foot posting is 3° varus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the rear foot posting is 3° valgus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the rear foot posting is 4° varus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the rear foot posting is 4° valgus foot position.

Preferably the method further includes determining a j-pod position, wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the j-pod position is neutral; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the j-pod position extends high laterally;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the j-pod position extends high medially;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the j-pod position extends high laterally;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the j-pod position extends high medially;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the j-pod position extends high laterally.

Preferably the method further includes determining a functional heel raise, wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the functional heel raise is substantially 0 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the functional heel raise is substantially 6 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the functional heel raise is substantially 6 mm.

Preferably the method includes providing a sole with the determined rear foot posting.

In one embodiment, the method includes making a sole with the determined rear foot posting.

Preferably the sole has an inherent heel raise of between 10 mm and 25 mm.

More preferably the sole has an inherent heel raise of between 15 mm and 20 mm.

Preferably the sole has a rocker radii angle of between 120 mm and 200 mm.

More preferably the sole has a rocker radii angle of substantially 160 mm.

Preferably the sole has a rocker radii position of between 40 to 70 percent of the sole length. More preferably the sole has a rocker radii position of substantially 60 percent of the sole length.

In another embodiment, the method includes providing footwear with the determined rear foot posting.

In one embodiment, the method includes making footwear with the determined rear foot posting.

It will be understood that in the embodiments including footwear, the footwear preferably includes the sole.

Preferably the footwear has an inherent heel raise of between 10 mm and 25 mm. More preferably the footwear has an inherent heel raise of between 15 mm and 20 mm.

Preferably the footwear has a rocker radii angle of between 120 mm and 200 mm. More preferably the footwear has a rocker radii angle of substantially 160 mm. Preferably the footwear has a rocker radii position of between 40 to 70 percent of the sole length. More preferably the footwear has a rocker radii position of substantially 60 percent of the sole length.

In another aspect, the present invention broadly resides in a method of determining a j-pod position of a sole, the method including the steps of:

determining a talar head palpation of a user's foot, wherein:

if the position of talus head is palpable on the lateral of the foot but not on the medial side, then the talar head palpation value receives a relative value of −2;

if the talar head is felt on the lateral of the foot and only slightly palpable on the medial side of the foot, then the talar head palpation value receives a relative value of −1;

if the talar head is equally palpable on the lateral and medial side of the foot, then the talar head palpation value receives a relative value of 0;

if the talar head is slightly palpable on lateral side and palpable on medial side, then the talar head palpation value receives a relative value of 1;

if the talar head is not palpable on lateral side, but palpable on medial side, then the talar head palpation value receives a relative value of 2; determining a supra and infra lateral malleoli curvature value of the user's foot,

wherein:

if the curve below the malleolus is either straight or convex, then the supra and infra lateral malleoli curvature value receives a relative value of −2;

if the curve below the malleolus is concave, but flatter or more than the curve above the malleolus, then the supra and infra lateral malleoli curvature value receives a relative value of −1;

if both the infra and supra malleolar curves are substantially equal, then the supra and infra lateral malleoli curvature value receives a relative value of 0;

if the curve below the malleolus is more concave than the curve above the malleolus, then the supra and infra lateral malleoli curvature value receives a relative value of 1;

if the curve below the malleolus is markedly more concave than the curve above the malleolus, then the supra and infra lateral malleoli curvature value receives a relative value of 2;

determining a calcaneal frontal plane position value of the user's foot, wherein:

if the calcaneus is more than 5° inverted, then the calcaneal frontal plane position value receives a relative value of −2;

if the calcaneus is between vertical and 5° inverted, then the calcaneal frontal plane position value receives a relative value of −1;

if the calcaneus is substantially vertical, then the calcaneal frontal plane position value receives a relative value of 0;

if the calcaneus is between vertical and 5° everted, then the calcaneal frontal plane position value receives a relative value of 1;

if the calcaneus is more than 5° everted, then the calcaneal frontal plane position value receives a relative value of 2;

determining a prominence of the talo-navicular joint value of the user's foot, wherein:

if the area of the talo-navicular joint is markedly concave, then the prominence of the talo-navicular joint value receives a relative value of −2;

if the area of the talo-navicular joint is slightly but definitely concave, then the prominence of the talo-navicular joint value receives a relative value of −1;

if the area of the talo-navicular joint is significantly flat, then the prominence of the talo-navicular joint value receives a relative value of 0;

if the area of the talo-navicular joint is bulging slightly, then the prominence of the talo-navicular joint value receives a relative value of 1;

if the area of the talo-navicular joint is bulging markedly, then the prominence of the talo-navicular joint value receives a relative value of 2;

determining a congruence of medial longitudinal arch value of the user's foot,

wherein:

if the arch is high and acutely angled towards the posterior end of the medial arch, then the congruence of medial longitudinal arch value receives a relative value of −2;

if the arch is moderately high and slightly acute posteriorly, then the congruence of medial longitudinal arch value receives a relative value of −1;

if the arch height is normal and concentrically curved, then the congruence of medial longitudinal arch value receives a relative value of 0;

if the arch is lowered with some flattening in the central position, then the congruence of medial longitudinal arch value receives a relative value of 1;

if the arch very low with severe flattening in the central portion and the arch makes ground contact, then the congruence of medial longitudinal arch value receives a relative value of 2;

determining an abduction/adduction of forefoot on rearfoot value of the user's foot, by viewing the foot from behind, wherein:

if there are no lateral toes visible and medial toes are clearly visible, then the abduction/adduction of forefoot on rearfoot value receives a relative value of −2;

if the medial toes are clearly more visible than the lateral toes, then the abduction/adduction of forefoot on rearfoot value receives a relative value of −1;

if the medial and lateral toes are equally visible, then the abduction/adduction of forefoot on rearfoot value receives a relative value of 0; if the lateral toes are clearly more visible than the medial toes, then the abduction/adduction of forefoot on rearfoot value receives a relative value of 1;

if no medial toes are visible and the lateral toes are clearly visible, then the abduction/adduction of forefoot on rearfoot value receives a relative value of 2;

wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the j-pod position is neutral; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the j-pod position extends high laterally;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the j-pod position extends high medially;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the j-pod position extends high laterally;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the j-pod position extends high medially;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the j-pod position extends high laterally.

Preferably the method further includes determining a rear foot posting, wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the rear foot posting is 0°;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the rear foot posting is 2° valgus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the rear foot posting is 3° varus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the rear foot posting is 3° valgus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the rear foot posting is 4° varus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the rear foot posting is 4° valgus foot position.

Preferably the method further includes determining a functional heel raise, wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the functional heel raise is substantially 0 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the functional heel raise is substantially 6 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the functional heel raise is substantially 6 mm.

Preferably the method includes providing a sole with the determined j-pod position.

In one embodiment, the method includes making a sole with the determined j-pod position.

Preferably the sole has an inherent heel raise of between 10 mm and 25 mm. More preferably the sole has an inherent heel raise of between 15 mm and 20 mm.

Preferably the sole has a rocker radii angle of between 120 mm and 200 mm. More preferably the sole has a rocker radii angle of substantially 160 mm.

Preferably the sole has a rocker radii position of between 40 to 70 percent of the sole length. More preferably the sole has a rocker radii position of substantially 60 percent of the sole length.

In another embodiment, the method includes providing footwear with the determined j-pod position.

In one embodiment, the method includes making footwear with the determined j-pod position.

It will be understood that in the embodiments including footwear, the footwear preferably includes the sole.

Preferably the footwear has an inherent heel raise of between 10 mm and 25 mm. More preferably the footwear has an inherent heel raise of between 15 mm and 20 mm.

Preferably the footwear has a rocker radii angle of between 120 mm and 200 mm. More preferably the footwear has a rocker radii angle of substantially 160 mm.

Preferably the footwear has a rocker radii position of between 40 to 70 percent of the sole length. More preferably the footwear has a rocker radii position of substantially 60 percent of the sole length.

In a further aspect, the present invention broadly resides in a method of determining a functional heel raise of a sole, the method including the steps of:

determining a talar head palpation of a user's foot, wherein:

if the position of talus head is palpable on the lateral of the foot but not on the medial side, then the talar head palpation value receives a relative value of −2;

if the talar head is felt on the lateral of the foot and only slightly palpable on the medial side of the foot, then the talar head palpation value receives a relative value of −1;

if the talar head is equally palpable on the lateral and medial side of the foot, then the talar head palpation value receives a relative value of 0;

if the talar head is slightly palpable on lateral side and palpable on medial side, then the talar head palpation value receives a relative value of 1;

if the talar head is not palpable on lateral side, but palpable on medial side, then the talar head palpation value receives a relative value of 2;

determining a supra and infra lateral malleoli curvature value of the user's foot, wherein:

if the curve below the malleolus is either straight or convex, then the supra and infra lateral malleoli curvature value receives a relative value of −2;

if the curve below the malleolus is concave, but flatter or more than the curve above the malleolus, then the supra and infra lateral malleoli curvature value receives a relative value of −1;

if both the infra and supra malleolar curves are substantially equal, then the supra and infra lateral malleoli curvature value receives a relative value of 0;

if the curve below the malleolus is more concave than the curve above the malleolus, then the supra and infra lateral malleoli curvature value receives a relative value of 1;

if the curve below the malleolus is markedly more concave than the curve above the malleolus, then the supra and infra lateral malleoli curvature value receives a relative value of 2;

determining a calcaneal frontal plane position value of the user's foot, wherein: if the calcaneus is more than 5° inverted, then the calcaneal frontal plane

position value receives a relative value of −2;

if the calcaneus is between vertical and 5° inverted, then the calcaneal frontal plane position value receives a relative value of −1;

if the calcaneus is substantially vertical, then the calcaneal frontal plane position value receives a relative value of 0;

if the calcaneus is between vertical and 5° everted, then the calcaneal frontal plane position value receives a relative value of 1;

if the calcaneus is more than 5° everted, then the calcaneal frontal plane position value receives a relative value of 2;

determining a prominence of the talo-navicular joint value of the user's foot, wherein:

if the area of the talo-navicular joint is markedly concave, then the prominence of the talo-navicular joint value receives a relative value of −2;

if the area of the talo-navicular joint is slightly but definitely concave, then the prominence of the talo-navicular joint value receives a relative value of −1;

if the area of the talo-navicular joint is significantly flat, then the prominence of the talo-navicular joint value receives a relative value of 0;

if the area of the talo-navicular joint is bulging slightly, then the prominence of the talo-navicular joint value receives a relative value of 1;

if the area of the talo-navicular joint is bulging markedly, then the prominence of the talo-navicular joint value receives a relative value of 2; determining a congruence of medial longitudinal arch value of the user's foot,

wherein:

if the arch is high and acutely angled towards the posterior end of the medial arch, then the congruence of medial longitudinal arch value receives a relative value of −2;

if the arch is moderately high and slightly acute posteriorly, then the congruence of medial longitudinal arch value receives a relative value of −1;

if the arch height is normal and concentrically curved, then the congruence of medial longitudinal arch value receives a relative value of 0;

if the arch is lowered with some flattening in the central position, then the congruence of medial longitudinal arch value receives a relative value of 1;

if the arch very low with severe flattening in the central portion and the arch makes ground contact, then the congruence of medial longitudinal arch value receives a relative value of 2;

determining an abduction/adduction of forefoot on rearfoot value of the user's foot, by viewing the foot from behind, wherein:

if there are no lateral toes visible and medial toes are clearly visible, then the abduction/adduction of forefoot on rearfoot value receives a relative value of −2;

if the medial toes are clearly more visible than the lateral toes, then the abduction/adduction of forefoot on rearfoot value receives a relative value of −1;

if the medial and lateral toes are equally visible, then the abduction/adduction of forefoot on rearfoot value receives a relative value of 0;

if the lateral toes are clearly more visible than the medial toes, then the abduction/adduction of forefoot on rearfoot value receives a relative value of 1;

if no medial toes are visible and the lateral toes are clearly visible, then the abduction/adduction of forefoot on rearfoot value receives a relative value of 2;

wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the functional heel raise is substantially 0 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the functional heel raise is substantially 4 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the functional heel raise is substantially 6 mm;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the functional heel raise is substantially 6 mm.

Preferably the method further includes determining a j-pod position, wherein

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the j-pod position is neutral; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the j-pod position extends high laterally;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the j-pod position extends high medially;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the j-pod position extends high laterally;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the j-pod position extends high medially;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the j-pod position extends high laterally.

Preferably the method further includes determining a rear foot posting, wherein if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the rear foot posting is 0°; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the rear foot posting is 2° valgus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the rear foot posting is 3° varus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the rear foot posting is 3° valgus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the rear foot posting is 4° varus foot position;

if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the rear foot posting is 4° valgus foot position.

Preferably the method includes providing a sole with the determined functional heel raise.

In one embodiment, the method includes making a sole with the determined functional heel raise.

Preferably the sole has an inherent heel raise of between 10 mm and 25 mm.

More preferably the sole has an inherent heel raise of between 15 mm and 20 mm.

Preferably the sole has a rocker radii angle of between 120 mm and 200 mm. More preferably the sole has a rocker radii angle of substantially 160 mm.

Preferably the sole has a rocker radii position of between 40 to 70 percent of the sole length. More preferably the sole has a rocker radii position of substantially 60 percent of the sole length.

In another embodiment, the method includes providing footwear with the determined functional heel raise.

In one embodiment, the method includes making footwear with the determined functional heel raise.

It will be understood that in the embodiments including footwear, the footwear preferably includes the sole.

Preferably the footwear has an inherent heel raise of between 10 mm and 25 mm. More preferably the footwear has an inherent heel raise of between 15 mm and 20 mm.

Preferably the footwear has a rocker radii angle of between 120 mm and 200 mm. More preferably the footwear has a rocker radii angle of substantially 160 mm.

Preferably the footwear has a rocker radii position of between 40 to 70 percent of the sole length. More preferably the footwear has a rocker radii position of substantially 60 percent of the sole length.

In one embodiment, the method further includes 3d printing a sole with one or more of the one or more attributes.

In one embodiment the step of determining a talar head palpation of a user's foot and assigning a value based on the position of the talus head includes using augmented reality. In another embodiment, the step of determining a talar head palpation of a user's foot and assigning a value based on the position of the talus head includes using machine learning to assign the value. Preferably the value is assigned using a scan or photo of the user's foot.

In one embodiment the step of determining a supra and infra lateral malleoli curvature of the user's foot and assigning a value based on the prominence of the infra and supra malleolar curves includes using augmented reality. In another embodiment, the step of determining a supra and infra lateral malleoli curvature of the user's foot and assigning a value based on the prominence of the infra and supra malleolar curves includes using machine learning to assign the value. Preferably the value is assigned using a scan or photo of the user's foot.

In one embodiment the step of determining a calcaneal frontal plane position of the user's foot and assigning a value based on an angle of the calcaneus includes using augmented reality. In another embodiment, the step of determining a calcaneal frontal plane position of the user's foot and assigning a value based on an angle of the calcaneus includes using machine learning to assign the value. Preferably the value is assigned using a scan or photo of the user's foot.

In one embodiment the step of determining a prominence of the talo-navicular joint of the user's foot and assigning a value based on the curvature of the talo-navicular joint includes using augmented reality. In another embodiment, the step of determining a prominence of the talo-navicular joint of the user's foot and assigning a value based on the curvature of the talo-navicular joint includes using machine learning to assign the value. Preferably the value is assigned using a scan or photo of the user's foot.

In one embodiment the step of determining a congruence of medial longitudinal arch of the user's foot and assigning a value based on the curvature of the arch includes using augmented reality. In another embodiment, the step of determining a congruence of medial longitudinal arch of the user's foot and assigning a value based on the curvature of the arch includes using machine learning to assign the value. Preferably the value is assigned using a scan or photo of the user's foot.

In one embodiment the step of determining an abduction/adduction of forefoot on rearfoot of the user's foot and assigning a valued based on the alignment includes using augmented reality. In another embodiment, the step of determining an abduction/adduction of forefoot on rearfoot of the user's foot and assigning a valued based on the alignment includes using machine learning to assign the value.

Preferably the value is assigned using a scan or photo of the user's foot. Preferably when determining a value for a feature of a user's foot, the value is one of five values assigned from one extreme condition for a feature to another extreme condition for a feature. Preferably, the values range from a first extreme condition, to a first moderate condition, to a neutral condition to a second moderate condition to a second extreme condition. In one embodiment, the values substantially correspond to different bands of a bell curve distribution for conditions. Preferably machine learning utilises a bell curve distribution or the like to assign values.

Preferably, if the method utilises augmented reality, a visual of a range of conditions from a plurality of ranges can be overlaid over the user's foot and a corresponding value can be assigned depending on which range the condition of the user's foot corresponds to.

In another aspect, the present invention broadly resides in a sole for a shoe, the sole made in accordance with a method as described in this specification.

In a further aspect, the present invention broadly resides in footwear made in accordance with a method as described in this specification.

The features described with respect to one aspect also apply where applicable to all other aspects of the invention. Furthermore, different combinations of described features are herein described and claimed even when not expressly stated.

### BRIEF DESCRIPTION OF THE DRAWINGS

In order that the present invention can be more readily understood reference will now be made to the accompanying drawings which illustrate a preferred embodiment of the invention and wherein:

FIG. 1 is a table listing conditions of a user's foot and corresponding values according to an embodiment of the present invention;

FIG. 2 is a table listing six bands of the possible sums of the assigned values;

FIG. 3 is a table listing attributes of a sole corresponding to the sum of assigned values;

FIG. 4 is a side view showing the inherent heel rise of a sole;

FIG. 5 is a side view showing the rocker radii position of a sole;

FIG. 6 is a side view showing the rocker radii angle of the sole of FIG. 5;

FIG. 7 is rear view showing a rear foot posting varus foot position;

FIG. 8 is a rear view showing a rear foot posting valgus foot position;

FIG. 9 is a side view showing the functional heel rise of a sole;

FIG. 10 is top view showing soles with a jpod position which extends laterally;

FIG. 11 is top view showing soles with a jpod position which extends medially; and

FIG. 12 is top view showing soles with a neutral jpod position.

### DETAILED DESCRIPTION OF THE PREFERRED EMBODIMENT

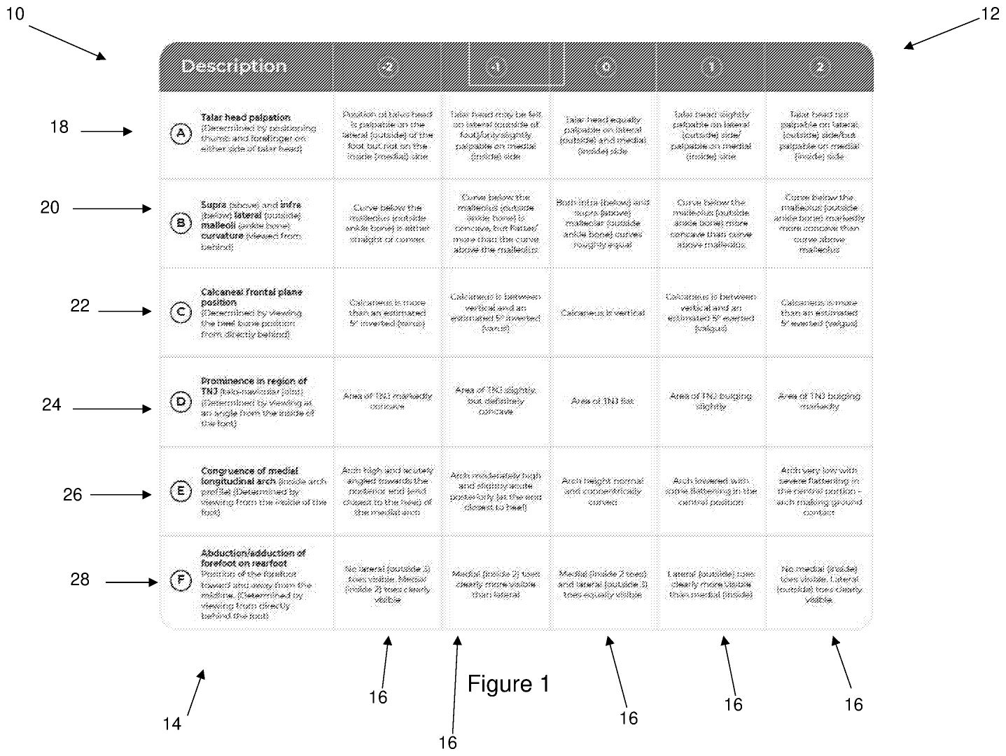

With reference to FIG. 1 there is shown a table 10 of the features 14 of a user's foot which are to be determined, possible results 16, and corresponding values 12 which are to be assigned.

The features 14 include talar head palpation 18, supra and infra lateral malleoli curvature 20, calcaneal frontal plane position 22, prominence of the talo-navicular joint 24, congruence of medial longitudinal arch 26, and abduction/adduction of forefoot on rearfoot 28.

The talar head palpation 18 can be determined by a person skilled in the art by using their index finger and thumb to palpate for the inside and outside of the talus head. The thumb is typically placed on the inside, and the index finger on the outside so that the person skilled in the art can ‘feel’ as to which side is more prominent. Columns 16 in row 18 give the possible results. When using augmented reality, possible results can be overlaid onto the user's foot and the person skilled in the art can choose the possible result which most closely matches the feature of the user's foot. When using machine learning, the possible result which most closely corresponds to the feature of the user's foot is used.

The supra and infra lateral malleoli curvature 20 can be determined by a person skilled in the art by viewing (from behind) the curve above and below the malleolus. Columns 16 in row 20 give the possible results. When using augmented reality, possible results can be overlaid onto the user's foot and the person skilled in the art can choose the possible result which most closely matches the feature of the user's foot. When using machine learning, the possible result which most closely corresponds to the feature of the user's foot is used.

The calcaneal frontal plane position 22 can be determined by a person skilled in the art by viewing from behind, the position of the calcaneus. Columns 16 in row 22 give the possible results. When using augmented reality, possible results can be overlaid onto the user's foot and the person skilled in the art can choose the possible result which most closely matches the feature of the user's foot. When using machine learning, the possible result which most closely corresponds to the feature of the user's foot is used.

The prominence of the talo-navicular joint 24 can be determined by a person skilled in the art by viewing the inside of the foot, and checking for the position of the talo-navicular joint. Columns 16 in row 24 give the possible results. When using augmented reality, possible results can be overlaid onto the user's foot and the person skilled in the art can choose the possible result which most closely matches the feature of the user's foot. When using machine learning, the possible result which most closely corresponds to the feature of the user's foot is used.

The congruence of medial longitudinal arch 26 can be determined by a person skilled in the art by viewing the inside of the foot, and inspecting the height of the arch profile. Columns 16 in row 26 give the possible results. When using augmented reality, possible results can be overlaid onto the user's foot and the person skilled in the art can choose the possible result which most closely matches the feature of the user's foot. When using machine learning, the possible result which most closely corresponds to the feature of the user's foot is used.

The abduction/adduction of forefoot on rearfoot 28 can be determined by a person skilled in the art by viewing the foot from behind and assessing if the forefoot is more prominent on the inside or outside of the foot. Columns 16 in row 28 give the possible results. When using augmented reality, possible results can be overlaid onto the user's foot and the person skilled in the art can choose the possible result which most closely matches the feature of the user's foot. When using machine learning, the possible result which most closely corresponds to the feature of the user's foot is used.

Row 12 gives the assigned values which correspond to the possible results in columns 16 and rows 18,20,22,24,26,28.

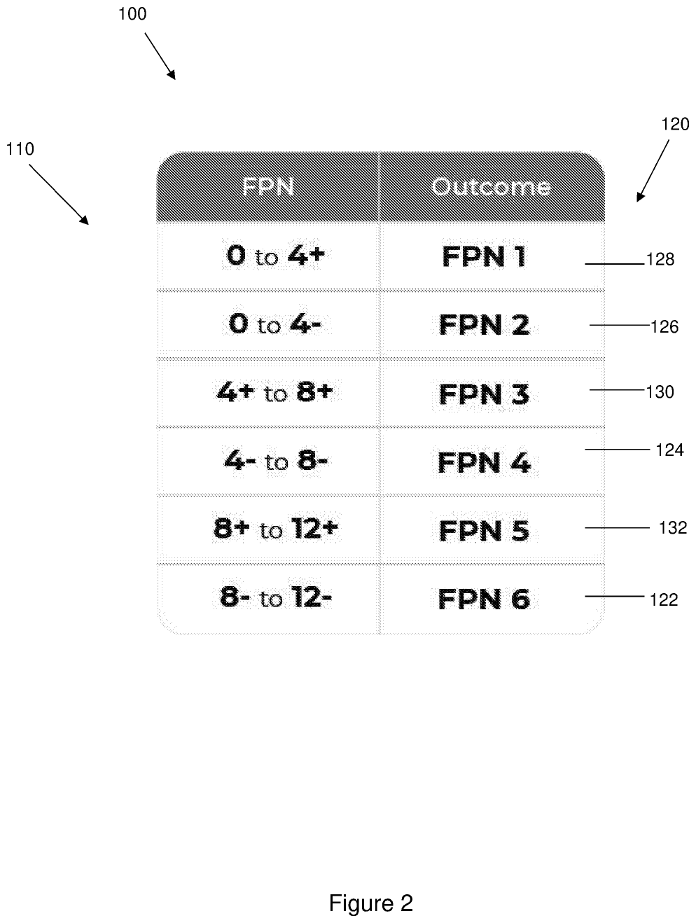

With reference to FIG. 2 there is shown a table 100 of the sum of the assigned values 110 and the corresponding bands 120.

The sum of values 110 is obtained by adding the assigned values (12 in FIG. 1) for the results for each feature (14 in FIG. 1).

FPN6 122 corresponds to band one. FPN4 124 corresponds to band two. FPN2 126 corresponds to band three. FPN1 128 corresponds to band four. FPN3 130 corresponds to band five. FPN5 corresponds to band six.

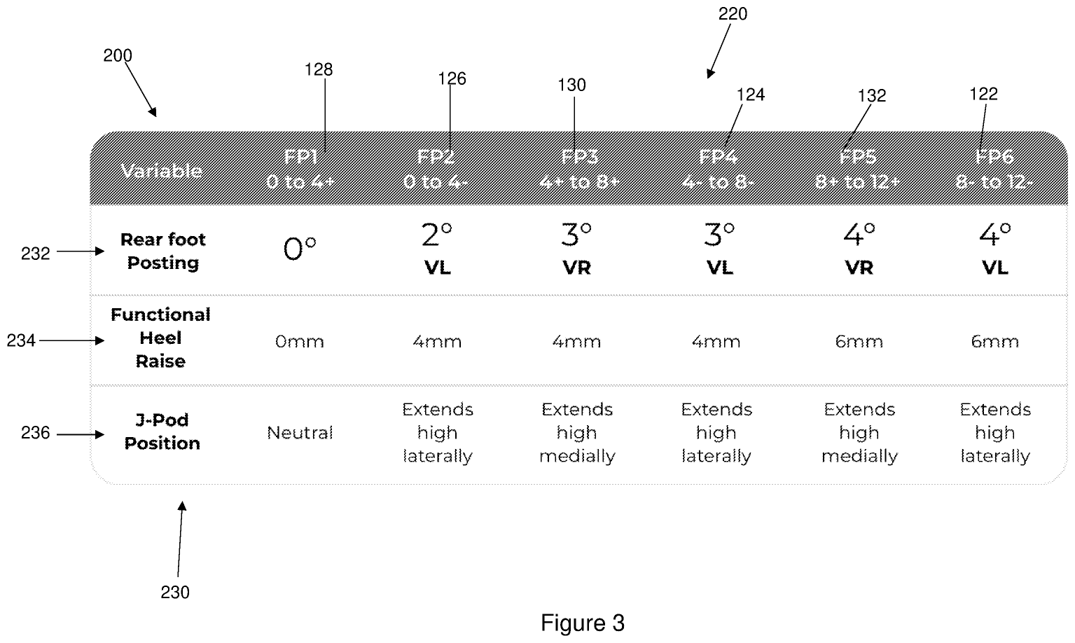

With reference to FIG. 3, there is shown a table 200 listing attributes of a sole. The table columns 220 corresponding to bands 122, 124, 126, 128, 130, 132 (as shown in FIG. 2). Column 230 lists the attributes to be determined. Rows 232,234,236 list the results of the attributes based on which band a user's foot corresponds with. For example, if a user's foot corresponds with band three 126 (FP2), then the attribute for rear foot posting 232 would be 2° valgus foot position, the attribute for functional heel raise 234 would be 4 mm, and the attribute for jpod position would be extends high laterally.

Using these attributes 232,234,236 for the corresponding bands 122, 124, 126, 128,130,132, a sole can be provide or made having one or more of the corresponding attributes 232,234,236.

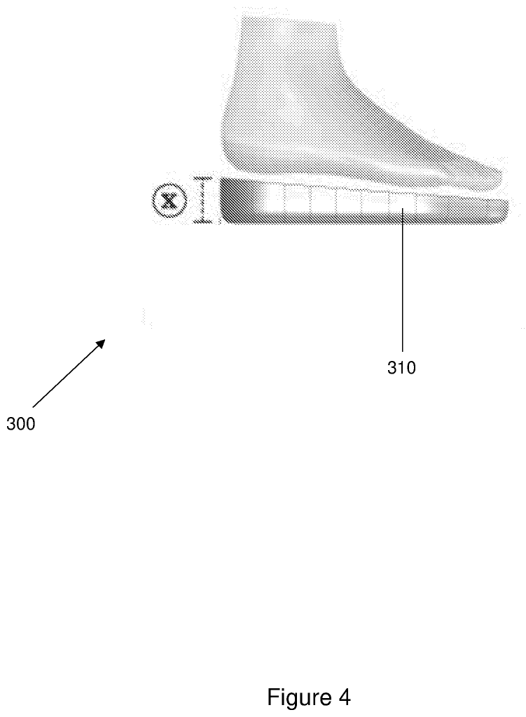

With reference to FIG. 4, there is shown where the inherent heel raise 300 is measured for a sole 310. Typically for a sole provided or manufactured (according to table 200 in FIG. 3), the inherent heel raise is between 15 mm and 20 mm.

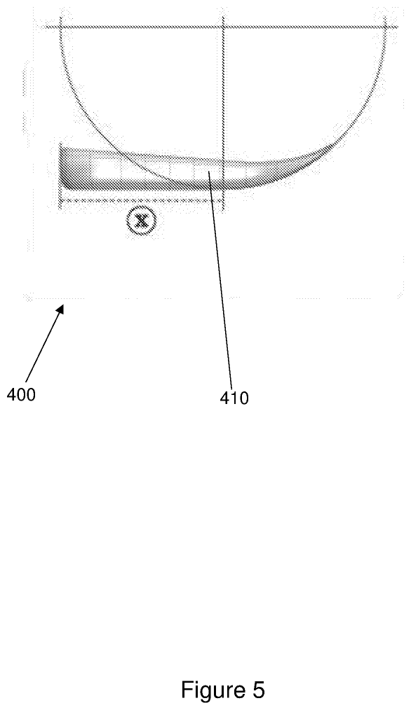

With reference to FIG. 5, there is shown where the rocker radii position 400 as a percentage of the length of a sole 410 is measured. Typically for a sole provided or manufactured (according to table 200 in FIG. 3), the rocker radii position 400 is around 60% of the length.

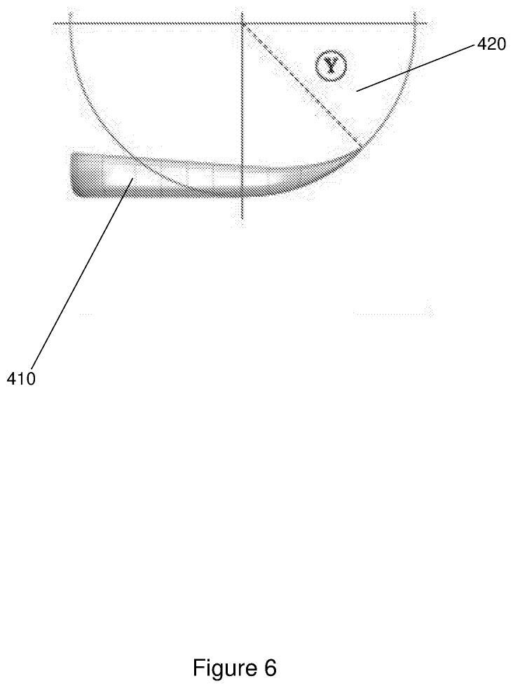

With reference to FIG. 6, there is shown where the rocker radii angle 420 of a sole 410 is measured. The rocker radii angle 420 is expressed as a radius. Typically for a sole provided or manufactured (according to table 200 in FIG. 3), the rocker radii angle 420 is around 160 mm.

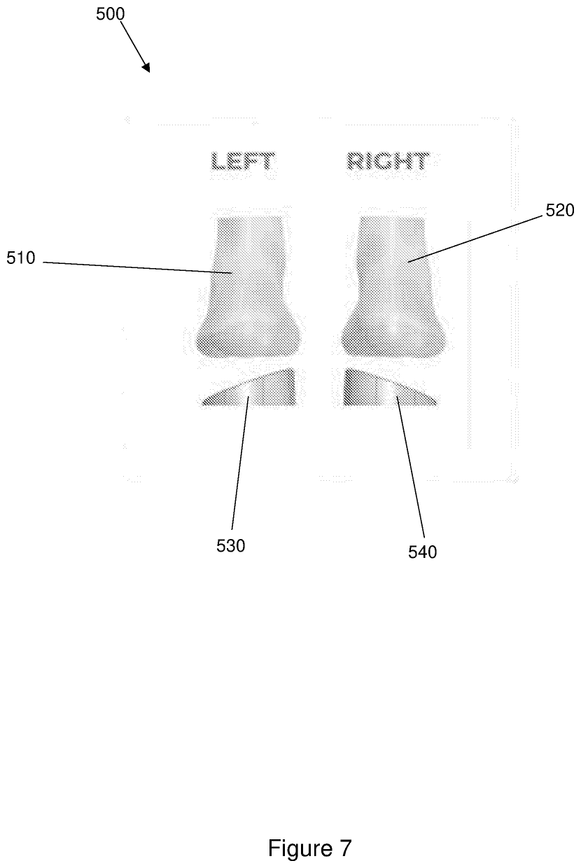

With reference to FIG. 7 there is shown a rear foot posting varus foot position 500, looking from the rear at a user's left foot 510 and right foot 520 and the corresponding soles 530,540.

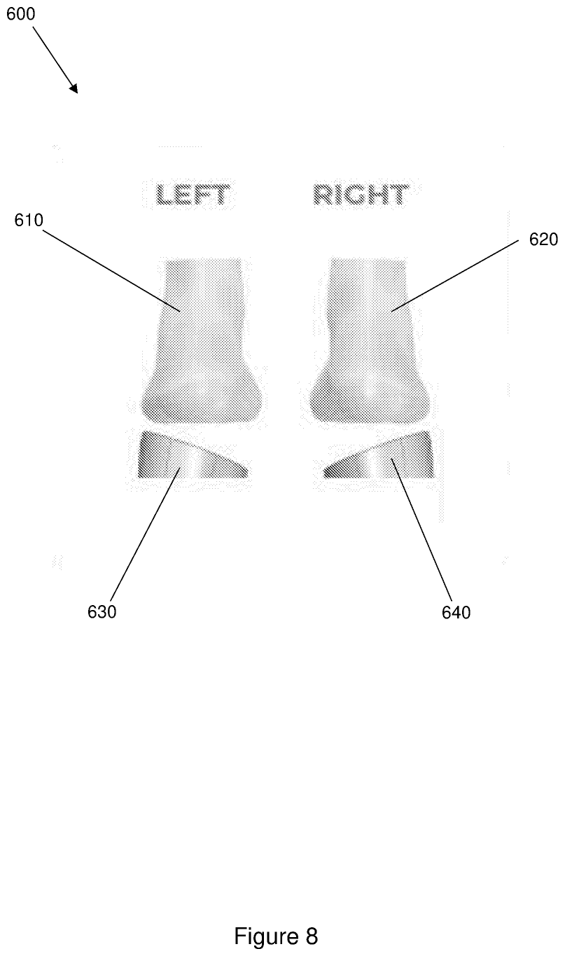

With reference to FIG. 8 there is shown a rear foot posting valgus foot position 600, looking from the rear at a user's left foot 610 and right foot 620 and the corresponding soles 630,640.

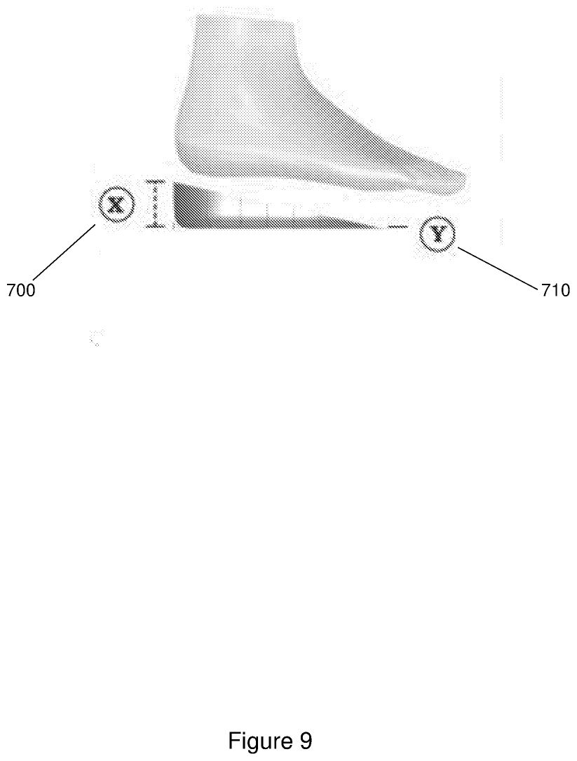

With reference to FIG. 9, there is shown where the functional heel raise 700 is measured with reference to a datum point 710.

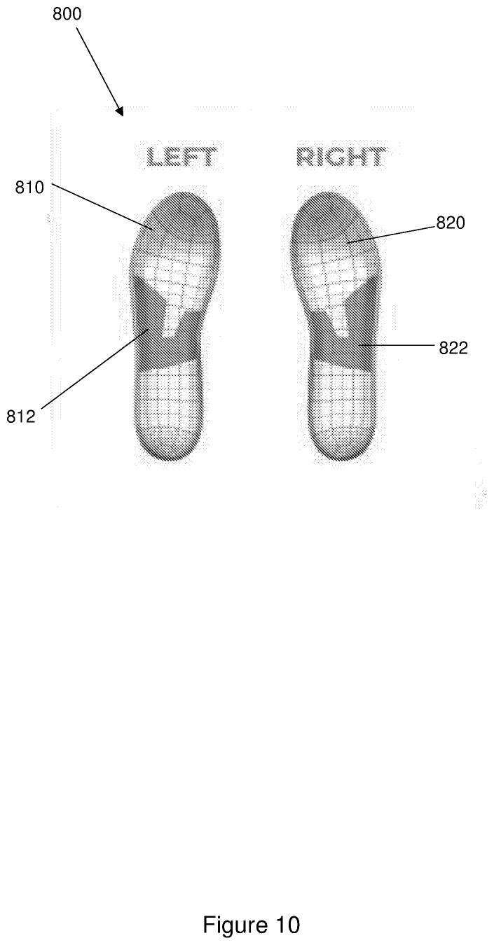

With reference to FIG. 10 there are shown jpod positions which extend laterally 800. On the left sole 810, the jpod position 812 extends laterally, and on the right sole 820 the jpod position 822 extends laterally.

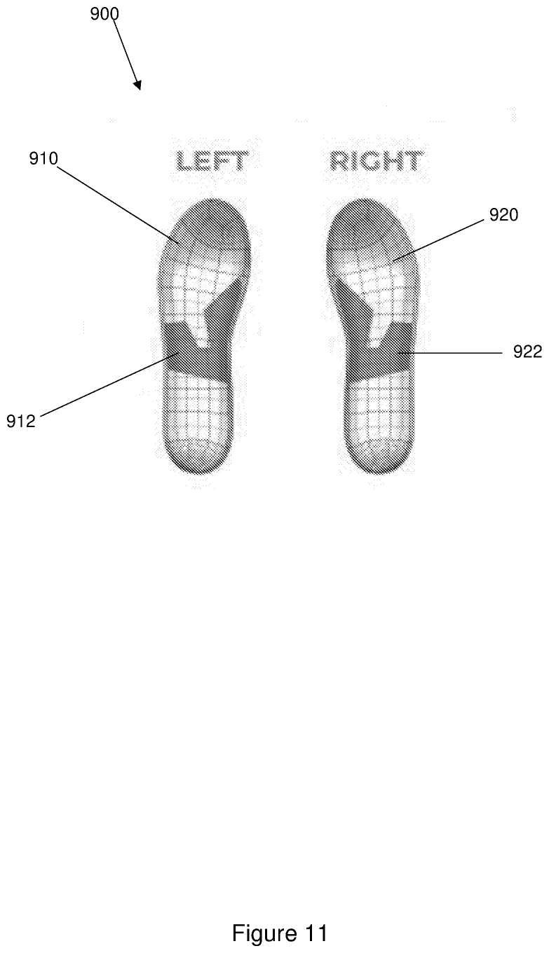

With reference to FIG. 11 there are shown jpod positions which extend medially 900. On the left sole 910, the jpod position 912 extends medially, and on the right sole 920 the jpod position 922 extends medially.

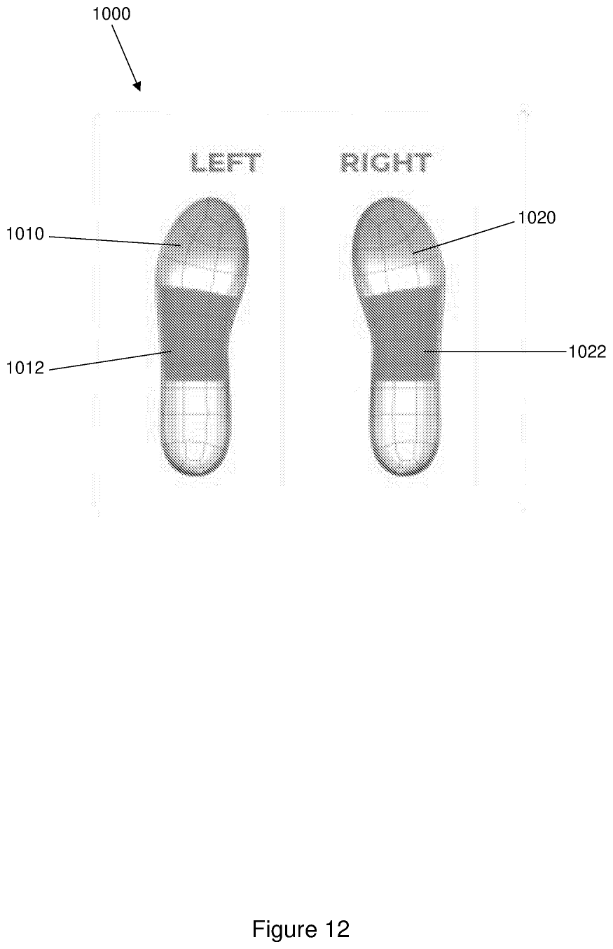

With reference to FIG. 12 there are shown neutral jpod positions 1000. On the left sole 1010, the jpod position 1012 is neutral, and on the right sole 1020 the jpod position 1022 is neutral.

### ADVANTAGES

An advantage of the preferred embodiment of the method of determining one or more attributes of a sole includes the ability to easily determine and provide an appropriate sole for a user. Another advantage of the preferred embodiment of the method of determining one or more attributes of a sole includes the ability to standardise attributes of a sole enabling standardisation of custom soles. A further advantage of the preferred embodiment of the method of determining one or more attributes of a sole includes the ability to mass produce footwear with customised soles.

### VARIATIONS

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A method of determining one or more attributes of a sole, the method including the steps of:
determining a talar head palpation of a user's foot and assigning a value based on the position of the talus head;
determining a supra and infra lateral malleoli curvature of the user's foot and assigning a value based on the prominence of the infra and supra malleolar curves;
determining a calcaneal frontal plane position of the user's foot and assigning a value based on an angle of the calcaneus;
determining a prominence of the talo-navicular joint of the user's foot and assigning a value based on the curvature of the talo-navicular joint;
determining a congruence of medial longitudinal arch of the user's foot and assigning a value based on the curvature of the arch;
determining an abduction/adduction of forefoot on rearfoot of the user's foot and assigning a value based on the alignment; and
determining a minimum possible sum of the values and a maximum possible sum of the values and dividing the range of values into a predetermined number of bands from the minimum possible sum to the maximum possible sum, wherein the one or more attributes is dependent on which band the sum of the assigned values fall into.

2. A method as claimed in claim 1 wherein the predetermined number of bands is six bands.

3. A method as claimed in claim 2, wherein one of the one or more attributes includes a rear foot posting varying between a 4° valgus foot position and a 4° varus foot position dependent on which band the sum of the assigned values fall into, wherein the rear foot posting is 4° valgus foot position if the sum of the assigned values falls into the first band, the rear foot posting is 3° valgus foot position if the sum of the assigned values falls into the second band, the rear foot posting is 2° valgus foot position if the sum of the assigned values falls into the third band, the rear foot posting is 0° if the sum of the assigned values falls into the fourth band, the rear foot posting is 3° varus foot position if the sum of the assigned values falls into the fifth band, and the rear foot posting is 4° varus foot position if the sum of the assigned values falls into the sixth band.

4. A method as claimed in claim 2, wherein one of the one or more attributes includes a j-pod position varying between extending high laterally and extending high medially dependent on which band the sum of the assigned values fall into, wherein the j-pod position extends high laterally if the sum of the assigned values falls into the first band, the j-pod position extends high laterally if the sum of the assigned values falls into the second band, the j-pod position extends high laterally if the sum of the assigned values falls into the third band, the j-pod position is neutral if the sum of the assigned values falls into the fourth band, the j-pod position extends high medially if the sum of the assigned values falls into the fifth band, and the j-pod position extends high medially if the sum of the assigned values falls into the sixth band.

5. A method as claimed in claim 2, wherein one of the one or more attributes includes a functional heel raise varying between 0 mm and 5 mm dependent on which band the sum of the assigned values fall into, wherein the functional heel raise is 6 mm if the sum of the assigned values falls into the first band, the functional heel raise is 4 mm if the sum of the assigned values falls into the second band, the functional heel raise is 4 mm if the sum of the assigned values falls into the third band, the functional heel raise is 0 mm if the sum of the assigned values falls into the fourth band, the functional heel raise is 4 mm if the sum of the assigned values falls into the fifth band, and the functional heel raise is 6 mm if the sum of the assigned values falls into the sixth band.

6. A method as claimed in claim 1, further including the step of making a sole having one or more of the one or more attributes.

7. A method as claimed in claim 6, wherein the sole further includes an inherent heel raise of between 10 mm and 25 mm; or an inherent heel raise of between 15 mm and 20 mm.

8. A method as claimed in claim 6, wherein the sole further includes a rocker radii angle of between 120 mm and 200 mm; or a rocker radii angle of substantially 160 mm.

9. A method as claimed in claim 6, wherein the sole further includes a rocker radii position of between 40 to 70 percent of the sole length.

10. A method as claimed in claim 6, wherein the sole further includes a rocker radii position of substantially 60 percent of the sole length.

11. A method as claimed in claim 6, wherein the sole has a Shore A hardness of between 25 to 65.

12. A method as claimed in claim 1, further including the step of 3d printing the sole.

13. A method as claimed in claim 1, wherein the step of determining a talar head palpation, a supra and infra lateral malleoli curvature, a calcaneal frontal plane position, a prominence of the talo-navicular joint, a congruence of medial longitudinal arch, or an abduction/adduction of forefoot on rearfoot of a user's foot and assigning a value includes using augmented reality.

14. A method as claimed in claim 1, wherein the step of determining a talar head palpation, a supra and infra lateral malleoli curvature, a calcaneal frontal plane position, a prominence of the talo-navicular joint, a congruence of medial longitudinal arch, or an abduction/adduction of forefoot on rearfoot of a user's foot and assigning a value includes using machine learning to assign the value.

15. A method of determining a rear foot posting of a sole, the method including the steps of:
determining a talar head palpation of a user's foot, wherein:
if the position of talus head is palpable on the lateral of the foot but not on the medial side, then the talar head palpation value receives a relative value of −2;
if the talar head is felt on the lateral of the foot and only slightly palpable on the medial side of the foot, then the talar head palpation value receives a relative value of −1;
if the talar head is equally palpable on the lateral and medial side of the foot, then the talar head palpation value receives a relative value of 0;
if the talar head is slightly palpable on lateral side and palpable on medial side, then the talar head palpation value receives a relative value of 1;
if the talar head is not palpable on lateral side, but palpable on medial side, then the talar head palpation value receives a relative value of 2;
determining a supra and infra lateral malleoli curvature value of the user's foot, wherein:
if the curve below the malleolus is either straight or convex, then the supra and infra lateral malleoli curvature value receives a relative value of −2;
if the curve below the malleolus is concave, but flatter or more than the curve above the malleolus, then the supra and infra lateral malleoli curvature value receives a relative value of −1;
if both the infra and supra malleolar curves are substantially equal, then the supra and infra lateral malleoli curvature value receives a relative value of 0;
if the curve below the malleolus is more concave than the curve above the malleolus, then the supra and infra lateral malleoli curvature value receives a relative value of 1;
if the curve below the malleolus is markedly more concave than the curve above the malleolus, then the supra and infra lateral malleoli curvature value receives a relative value of 2;
determining a calcaneal frontal plane position value of the user's foot, wherein: if the calcaneus is more than 5° inverted, then the calcaneal frontal plane position value receives a relative value of −2;
if the calcaneus is between vertical and 5° inverted, then the calcaneal frontal plane position value receives a relative value of −1;
if the calcaneus is substantially vertical, then the calcaneal frontal plane position value receives a relative value of 0;
if the calcaneus is between vertical and 5° everted, then the calcaneal frontal plane position value receives a relative value of 1;
if the calcaneus is more than 5° everted, then the calcaneal frontal plane position value receives a relative value of 2;
determining a prominence of the talo-navicular joint value of the user's foot, wherein:
if the area of the talo-navicular joint is markedly concave, then the prominence of the talo-navicular joint value receives a relative value of −2;
if the area of the talo-navicular joint is slightly but definitely concave, then the prominence of the talo-navicular joint value receives a relative value of −1;
if the area of the talo-navicular joint is significantly flat, then the prominence of the talo-navicular joint value receives a relative value of 0;
if the area of the talo-navicular joint is bulging slightly, then the prominence of the talo-navicular joint value receives a relative value of 1;
if the area of the talo-navicular joint is bulging markedly, then the
prominence of the talo-navicular joint value receives a relative value of 2; determining a congruence of medial longitudinal arch value of the user's foot, wherein:
if the arch is high and acutely angled towards the posterior end of the medial arch, then the congruence of medial longitudinal arch value receives a relative value of −2;
if the arch is moderately high and slightly acute posteriorly, then the congruence of medial longitudinal arch value receives a relative value of −1;
if the arch height is normal and concentrically curved, then the congruence of medial longitudinal arch value receives a relative value of 0;
if the arch is lowered with some flattening in the central position, then the congruence of medial longitudinal arch value receives a relative value of 1;
if the arch very low with severe flattening in the central portion and the arch makes ground contact, then the congruence of medial longitudinal arch value receives a relative value of 2;
determining an abduction/adduction of forefoot on rearfoot value of the user's foot, by viewing the foot from behind, wherein:
if there are no lateral toes visible and medial toes are clearly visible, then the abduction/adduction of forefoot on rearfoot value receives a relative value of −2;
if the medial toes are clearly more visible than the lateral toes, then the abduction/adduction of forefoot on rearfoot value receives a relative value of −1;
if the medial and lateral toes are equally visible, then the abduction/adduction of forefoot on rearfoot value receives a relative value of 0;
if the lateral toes are clearly more visible than the medial toes, then the abduction/adduction of forefoot on rearfoot value receives a relative value of 1; if no medial toes are visible and the lateral toes are clearly visible, then
the abduction/adduction of forefoot on rearfoot value receives a relative value 5 of 2;
wherein
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the rear foot posting is 0°; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the rear foot posting is 2° valgus foot position;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the rear foot posting is 3° varus foot position;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the rear foot posting is 3° valgus foot position;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the rear foot posting is 4° varus foot position;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the rear foot posting is 4° valgus foot position.

16. A method of determining a j-pod position of a sole, the method including the steps of:
determining a talar head palpation of a user's foot, wherein:
if the position of talus head is palpable on the lateral of the foot but not on the medial side, then the talar head palpation value receives a relative value of −2;
if the talar head is felt on the lateral of the foot and only slightly palpable on the medial side of the foot, then the talar head palpation value receives a relative value of −1;
if the talar head is equally palpable on the lateral and medial side of the foot, then the talar head palpation value receives a relative value of 0;
if the talar head is slightly palpable on lateral side and palpable on medial side, then the talar head palpation value receives a relative value of 1;
if the talar head is not palpable on lateral side, but palpable on medial side, then the talar head palpation value receives a relative value of 2; determining a supra and infra lateral malleoli curvature value of the user's foot,
wherein:
if the curve below the malleolus is either straight or convex, then the supra and infra lateral malleoli curvature value receives a relative value of −2;
if the curve below the malleolus is concave, but flatter or more than the curve above the malleolus, then the supra and infra lateral malleoli curvature value receives a relative value of −1;
if both the infra and supra malleolar curves are substantially equal, then the supra and infra lateral malleoli curvature value receives a relative value of 0;
if the curve below the malleolus is more concave than the curve above the malleolus, then the supra and infra lateral malleoli curvature value receives a relative value of 1;
if the curve below the malleolus is markedly more concave than the curve above the malleolus, then the supra and infra lateral malleoli curvature value receives a relative value of 2;
determining a calcaneal frontal plane position value of the user's foot, wherein:
if the calcaneus is more than 5° inverted, then the calcaneal frontal plane position value receives a relative value of −2;
if the calcaneus is between vertical and 5° inverted, then the calcaneal frontal plane position value receives a relative value of −1;
if the calcaneus is substantially vertical, then the calcaneal frontal plane position value receives a relative value of 0;
if the calcaneus is between vertical and 5° everted, then the calcaneal frontal plane position value receives a relative value of 1;
if the calcaneus is more than 5° everted, then the calcaneal frontal plane position value receives a relative value of 2;
determining a prominence of the talo-navicular joint value of the user's foot, wherein:
if the area of the talo-navicular joint is markedly concave, then the prominence of the talo-navicular joint value receives a relative value of −2;
if the area of the talo-navicular joint is slightly but definitely concave, then the prominence of the talo-navicular joint value receives a relative value of −1;
if the area of the talo-navicular joint is significantly flat, then the prominence of the talo-navicular joint value receives a relative value of 0;
if the area of the talo-navicular joint is bulging slightly, then the prominence of the talo-navicular joint value receives a relative value of 1;
if the area of the talo-navicular joint is bulging markedly, then the prominence of the talo-navicular joint value receives a relative value of 2;
determining a congruence of medial longitudinal arch value of the user's foot,
wherein:
if the arch is high and acutely angled towards the posterior end of the medial arch, then the congruence of medial longitudinal arch value receives a relative value of −2;
if the arch is moderately high and slightly acute posteriorly, then the congruence of medial longitudinal arch value receives a relative value of −1;
if the arch height is normal and concentrically curved, then the congruence of medial longitudinal arch value receives a relative value of 0;
if the arch is lowered with some flattening in the central position, then the congruence of medial longitudinal arch value receives a relative value of 1;
if the arch very low with severe flattening in the central portion and the arch makes ground contact, then the congruence of medial longitudinal arch value receives a relative value of 2;
determining an abduction/adduction of forefoot on rearfoot value of the user's foot, by viewing the foot from behind, wherein:
if there are no lateral toes visible and medial toes are clearly visible, then the abduction/adduction of forefoot on rearfoot value receives a relative value of −2;
if the medial toes are clearly more visible than the lateral toes, then the abduction/adduction of forefoot on rearfoot value receives a relative value of −1;
if the medial and lateral toes are equally visible, then the abduction/adduction of forefoot on rearfoot value receives a relative value of 0; if the lateral toes are clearly more visible than the medial toes, then the abduction/adduction of forefoot on rearfoot value receives a relative value of 1;
if no medial toes are visible and the lateral toes are clearly visible, then the abduction/adduction of forefoot on rearfoot value receives a relative value of 2;
wherein
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the j-pod position is neutral; if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the j-pod position extends high laterally;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the j-pod position extends high medially;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the j-pod position extends high laterally;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the j-pod position extends high medially;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the j-pod position extends high laterally.

17. A method of determining a functional heel raise of a sole, the method including the steps of:
determining a talar head palpation of a user's foot, wherein:
if the position of talus head is palpable on the lateral of the foot but not on the medial side, then the talar head palpation value receives a relative value of −2;
if the talar head is felt on the lateral of the foot and only slightly palpable on the medial side of the foot, then the talar head palpation value receives a relative value of −1;
if the talar head is equally palpable on the lateral and medial side of the foot, then the talar head palpation value receives a relative value of 0;
if the talar head is slightly palpable on lateral side and palpable on medial side, then the talar head palpation value receives a relative value of 1;
if the talar head is not palpable on lateral side, but palpable on medial side, then the talar head palpation value receives a relative value of 2;
determining a supra and infra lateral malleoli curvature value of the user's foot, wherein:
if the curve below the malleolus is either straight or convex, then the supra and infra lateral malleoli curvature value receives a relative value of −2;
if the curve below the malleolus is concave, but flatter or more than the curve above the malleolus, then the supra and infra lateral malleoli curvature value receives a relative value of −1;
if both the infra and supra malleolar curves are substantially equal, then the supra and infra lateral malleoli curvature value receives a relative value of 0;
if the curve below the malleolus is more concave than the curve above the malleolus, then the supra and infra lateral malleoli curvature value receives a relative value of 1;
if the curve below the malleolus is markedly more concave than the curve above the malleolus, then the supra and infra lateral malleoli curvature value receives a relative value of 2;
determining a calcaneal frontal plane position value of the user's foot, wherein: if the calcaneus is more than 5° inverted, then the calcaneal frontal plane
position value receives a relative value of −2;
if the calcaneus is between vertical and 5° inverted, then the calcaneal frontal plane position value receives a relative value of −1;
if the calcaneus is substantially vertical, then the calcaneal frontal plane position value receives a relative value of 0;
if the calcaneus is between vertical and 5° everted, then the calcaneal frontal plane position value receives a relative value of 1;
if the calcaneus is more than 5° everted, then the calcaneal frontal plane position value receives a relative value of 2;
determining a prominence of the talo-navicular joint value of the user's foot, wherein:
if the area of the talo-navicular joint is markedly concave, then the prominence of the talo-navicular joint value receives a relative value of −2;
if the area of the talo-navicular joint is slightly but definitely concave, then the prominence of the talo-navicular joint value receives a relative value of −1;
if the area of the talo-navicular joint is significantly flat, then the prominence of the talo-navicular joint value receives a relative value of 0;
if the area of the talo-navicular joint is bulging slightly, then the prominence of the talo-navicular joint value receives a relative value of 1;
if the area of the talo-navicular joint is bulging markedly, then the prominence of the talo-navicular joint value receives a relative value of 2; determining a congruence of medial longitudinal arch value of the user's foot,
wherein:
if the arch is high and acutely angled towards the posterior end of the medial arch, then the congruence of medial longitudinal arch value receives a relative value of −2;
if the arch is moderately high and slightly acute posteriorly, then the congruence of medial longitudinal arch value receives a relative value of −1;
if the arch height is normal and concentrically curved, then the congruence of medial longitudinal arch value receives a relative value of 0;
if the arch is lowered with some flattening in the central position, then the congruence of medial longitudinal arch value receives a relative value of 1;
if the arch very low with severe flattening in the central portion and the arch makes ground contact, then the congruence of medial longitudinal arch value receives a relative value of 2;
determining an abduction/adduction of forefoot on rearfoot value of the user's foot, by viewing the foot from behind, wherein:
if there are no lateral toes visible and medial toes are clearly visible, then the abduction/adduction of forefoot on rearfoot value receives a relative value of −2;
if the medial toes are clearly more visible than the lateral toes, then the abduction/adduction of forefoot on rearfoot value receives a relative value of −1;
if the medial and lateral toes are equally visible, then the abduction/adduction of forefoot on rearfoot value receives a relative value of 0;
if the lateral toes are clearly more visible than the medial toes, then the abduction/adduction of forefoot on rearfoot value receives a relative value of 1;
if no medial toes are visible and the lateral toes are clearly visible, then the abduction/adduction of forefoot on rearfoot value receives a relative value of 2;
wherein
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 0 and 4, then the functional heel raise is substantially 0 mm;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −1 and −4, then the functional heel raise is substantially 4 mm;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 5 and 8, then the functional heel raise is substantially 4 mm;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −5 and −8, then the functional heel raise is substantially 4 mm;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between 9 and 12, then the functional heel raise is substantially 6 mm;
if the talar head palpation value, the infra lateral malleoli curvature value, the calcaneal frontal plane position value, the prominence of the talo-navicular joint value, the congruence of medial longitudinal arch value, and the abduction/adduction of forefoot on rearfoot value add up to between −9 and −12, then the functional heel raise is substantially 6 mm.

18. A sole for a shoe, the sole made in accordance with a method as claimed in claim 1.

19. Footwear having a sole as claimed in claim 1.

20. Footwear made in accordance with a method as claimed in claim 1.
